SEQUENCE LISTING (7 sequences)

SEQ ID NO: 1 - protein, 32 aa, Homo sapiens
  DISULFID (10)..(26)
SPKMVQGSGCFGRKMDRISSSSGLGCKVLRRH

SEQ ID NO: 2 - protein, 28 aa, Homo sapiens
  DISULFID (7)..(23)
SLRRSSCFGGRMDRIGAQSGLGCNSFRY

SEQ ID NO: 3 - protein, 30 aa, Homo sapiens
NPMYNAVSNADLMDFKNLLDHLEEKMPLED

SEQ ID NO: 4 - protein, 37 aa, Homo sapiens
EVVPPQVLSEPNEEAGAALSPLPEVPPWTGEVSPAQR

SEQ ID NO: 5 - protein, 19 aa, Homo sapiens
SDRSALLKSKLRALLTAPR

SEQ ID NO: 6 - protein, 28 aa, Homo sapiens
  DISULFID (7)..(23)
SLRRSSCFGGRMDRIGAQSGLGCNSFRY

SEQ ID NO: 7 - protein, 32 aa, Homo sapiens
TAPRSLRRSSCFGGRMDRIGAQSGLGCNSFRY

(19) United States
(12) Patent Application Publication
(10) Pub. No.: US 2015/0038418 A1
(43) Pub. Date: Feb. 5, 2015

(54) NATRIURETIC PEPTIDE COMPOSITIONS AND METHODS OF PREPARATION

(71) Applicants: William Van Antwerp, Valencia, CA (US); Dianne Judd, Minneapolis, MN (US); Andrew Walsh, Minneapolis, MN (US); Lian Luo, Plymouth, MN (US)

(72) Inventors: William Van Antwerp, Valencia, CA (US); Dianne Judd, Minneapolis, MN (US); Andrew Walsh, Minneapolis, MN (US); Lian Luo, Plymouth, MN (US)

(73) Assignee: MEDTRONIC, INC., Minneapolis, MN (US)

(21) Appl. No.: 14/235,364

(22) PCT Filed: Jul. 19, 2012
(86) PCT No.: PCT/US2012/047492

§ 371 Date: Mar. 27, 2014

Related U.S. Application Data
(60) Provisional application No. 61/512,369, filed on Jul. 27, 2011.

(51) Int. Cl. C07K 14/58

(52) U.S. Cl. CPC C07K 14/58; A61K 38/00
USPC 514/12.4

(57) ABSTRACT

Therapeutic compositions containing natriuretic peptides for treating chronic kidney disease alone, heart failure alone, or chronic kidney disease with concomitant heart failure are described. The therapeutic compositions have enhanced stability characteristics to facilitate storage and delivery by provisioning apparatuses under conditions of elevated temperature and mechanical stress. Methods for increasing the stability of therapeutic compositions containing natriuretic peptides are further described.

### REFERENCE TO SEQUENCE LISTING

This application contains a “Sequence Listing” submitted as an electronic .txt file. The information contained in the Sequence Listing is hereby incorporated by reference.

### FIELD OF THE INVENTION

The invention relates to stable compositions for the administration of natriuretic peptides used in the treatment of pathological conditions such as Chronic Kidney Disease (CKD) alone, heart failure alone, or with concomitant CKD and Heart Failure (HF). Methods for preparing the stable compositions and use thereof are also provided.

### BACKGROUND

Proteins and peptides serve multifunctional roles in biological systems. As ligands for various receptors as well as substrates for various enzymes, peptides can be used to regulate biological processes wherein the function of proteins and peptides can be defined by the structure, orientation and positioning of side-chains in aqueous solution as determined by secondary and tertiary structure. However, the primary sequence of amino acid residues needed for proper orientation in aqueous solution can sometimes lead to instability. Due to the difficulties in delivering peptide-based drugs, relatively few peptide-based drugs are currently on the market. Although insulin and insulin derivatives are one of the first commercially formulated peptide based drugs, insulin has several structural features allowing for insulin to remain stable during long storage periods in solution compared with small peptides and particularly compared with small peptides synthesized through solid-phase synthesis rather than recombinant methods expressed inside a living cell. Without being limited to any one theory, human insulin, including human insulin recombinantly expressed in bacterial cells, is formed of two separate peptide chains having 21 and 30 amino acid residues, respectively. The two peptide chains are linked through three disulfide bridges between pairs of cysteine residues. Both peptide chains form a significant amount of alpha-helical secondary structure. Due to the disulfide bridges linking the peptide chains, the alpha-helical regions of the two peptide chains contact one another forming numerous salt bridges and van der Waals contacts. As a result, insulin has a well-ordered tertiary structure that stabilizes insulin against surface adsorption by reducing the exposure of hydrophobic regions to a surrounding aqueous environment. Further, the structure of insulin reduces mobility of the peptide backbone helping to protect insulin from proteolytic attack from acids or bases. Insulin in solution can form hexamers mediated by zinc ions, which further stabilizes its structures. Many commercial formulations of insulin contain zinc salts to promote stability.

Natriuretic peptides have a structure allowing for binding to atrial natriuretic peptide (ANP) receptor, which controls the activity of an associated guanylyl cyclase. The binding of an agonist ligand to the ANP receptor results in several physiological effects including decrease in cardiac volume and blood output, decrease in blood pressure and increase in glomerular filtration rate (GFR). Without being limited to any single theory, the natriuretic peptides are believed to have certain amounts of unordered structure and/or loops that can undertake several conformations in solution. The presence of unordered regions along the peptide backbone could allow for a high degree of freedom of movement in the peptide chain, which can open the peptide chain to attack by proteolytic enzymes and acid/base attack as well as other chemical reactions such as deamidation. Further, hydrophobic regions of the peptide or regions that are susceptible to surface adsorption can be exposed to the environment. Hence, certain peptides and polypeptides, such as natriuretic peptides, may be rapidly degraded when formulated into a solution for administration. In particular, the amide bonds forming the peptide backbone can be subject to nucleophilic attack and hydrolysis in aqueous solutions. Further, peptides can be degraded by peptidases, amidases, and/or esterases present in the environment.

Stable formulations of therapeutic agents are important for use in delivery devices that expose peptides to elevated temperatures, mechanical stress and/or hydrophobic interactions with components of delivery devices. Formulations of peptides should remain soluble and substantially free of aggregation, even though subjected to the patient's body heat and motion for periods ranging from a few days to several months. Of the 20 amino acids that form most natural peptide sequences, many have side chains that are hydrophobic, where peptides containing a high amount of such hydrophobic amino acid residues may have limited solubility in aqueous solution or undergo aggregation over time. For this reason, some peptides may have limited therapeutic use. Even in situations where a peptide has pharmacological effect when administered, the concentration of the peptide in an aqueous pharmaceutical composition can be unstable. Depending on the particular administration requirements and time limitations, a formulation with a short shelf-life may have little practical value. While organic solvents increase the solubility of most peptides, the presence of organic solvents in compositions for injection can be problematic. Chemical modifications of peptides to increase solubility are also known. Such chemical modifications can take the form of substitution of specific amino acid residues as well as covalent attachment to the N- and/or C-terminus of groups serving to increase solubility. Without being limited to any particular theory, such chemical modification can undesirably decrease the biological efficacy of the peptide.

Hence, there is a need for a stable formulation of one or more natriuretic peptides having a long-shelf life that can be stably used in mechanical delivery or implantable devices for protracted periods of time at required temperatures. There is also a need for a method for preparing such stable natriuretic peptide formulations.

### SUMMARY OF THE INVENTION

The disclosure provided herein is directed to compositions for stabilizing aqueous solutions containing natriuretic peptides, such as Vessel Dilator (VD), during storage and administration to a patient and methods for preparing such stabilized solutions. The invention disclosed herein has a number of embodiments that relate to therapeutic methods and compositions for treatment of Chronic Kidney Disease (CKD) alone, Heart Failure (HF) alone or with concomitant CKD and HF.

The systems and methods of the invention are directed to the administration of a natriuretic peptide to a patient for the treatment of CKD alone, HF alone or with concomitant CKD and HF. The systems and methods of the invention are also useful for treating other renal or cardiovascular diseases, such as congestive heart failure (CHF), dyspnea, elevated pulmonary capillary wedge pressure, chronic renal insufficiency, acute renal failure, cardiorenal syndrome, contrast induced nephropathy (CIN) and diabetes mellitus.

In certain embodiments, a therapeutic protein composition contains a protein, peptide or polypeptide selected from the group consisting of vessel dilator (VD) and kaliuretic peptide (KP), from about 0.15% to about 0.315% of m-cresol by weight (3-methylphenol), tris(hydroxymethyl)aminomethane and water. The pH of the therapeutic protein composition can be from about 6.5 to 7.6.

In certain embodiments, the therapeutic protein composition has a pH from about 6.5 to 7.6 when the therapeutic protein composition is adjusted to a temperature of 25° C.

In certain embodiments, a concentration of tris(hydroxymethyl)aminomethane in the therapeutic protein composition is from about 5 to about 200 mM, from about 5 to about 100 mM or from about 10 to about 70 mM.

In certain embodiments, a concentration of phosphate buffer in the therapeutic composition is from about 0.2 to about 10 grams per liter.

In certain embodiments, the protein composition further comprises from about 0.1 to about 5% glycerol by weight.

In certain embodiments, a therapeutic protein composition is stored in a container. The protein composition contains a protein, peptide or polypeptide selected from the group consisting of vessel dilator (VD) and kaliuretic peptide (KP), from about 0.15% to about 0.315% of m-cresol by weight, and an aqueous (hydroxymethyl)aminomethane buffer. The therapeutic protein composition is administered and metered to a patient using a pump.

In certain embodiments, the therapeutic protein composition contains aqueous (hydroxymethyl)aminomethane and from about 0.15% to about 0.315% of m-cresol by weight, wherein the protein composition is formed at least 14 days prior to administration of the protein composition to a patient.

In certain embodiments, the therapeutic protein composition is stored in a container for a time period of at least 14 days and the amount of a protein, polypeptide or peptide present in the composition after 14 days is about 95% or more of the amount of the protein, polypeptide or peptide comprised in the therapeutic protein composition prior to storage in the container for 14 days.

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 shows the recovery of vessel dilator (VD) peptide after storage in solutions of tris-(hydroxymethyl)-aminomethane (Tris), phosphate buffered saline (PBS) and saline.

FIG. 2 shows the purity of vessel dilator (VD) peptide after storage in solutions of Tris, PBS and saline.

FIG. 3 shows the recovery of vessel dilator (VD) peptide at a concentration of 1.25 mg/mL in a Tris buffer delivered by a provisioning apparatus or stored in an unpumped reservoir.

FIG. 4 shows the recovery of vessel dilator (VD) peptide at a concentration of 1.25 mg/mL in Tris buffer after storage in a glass vial.

FIG. 5 shows the purity of vessel dilator (VD) peptide at a concentration of 1.25 mg/mL in Tris buffer during delivery from a provisioning apparatus or after storage in a glass vial.

FIG. 6 shows the recovery of vessel dilator (VD) peptide at a concentration of 1.25 mg/mL in PBS delivered by a provisioning apparatus or stored in an unpumped reservoir.

FIG. 7 shows the recovery of vessel dilator (VD) peptide at a concentration of 1.25 mg/mL in PBS after storage in a glass vial.

FIG. 8 shows the purity of vessel dilator (VD) peptide at a concentration of 1.25 mg/mL in PBS during delivery from a provisioning apparatus or after storage in a glass vial.

FIG. 9 shows the recovery of vessel dilator (VD) peptide at a concentration of 15 mg/mL in a Tris buffer delivered by a provisioning apparatus or stored in an unpumped reservoir.

FIG. 10 shows the purity of vessel dilator (VD) peptide at a concentration of 15 mg/mL in Tris buffer during delivery from a provisioning apparatus or after storage in a glass vial.

FIG. 11 shows the recovery of vessel dilator (VD) peptide at a concentration of 15 mg/mL in PBS delivered by a provisioning apparatus or stored in an unpumped reservoir.

FIG. 12 shows the purity of vessel dilator (VD) peptide at a concentration of 15 mg/mL in PBS during delivery from a provisioning apparatus or after storage in a glass vial.

FIG. 13 presents the concentration of Prostaglandin E₂ in blood serum of a rat model administered vessel dilator (VD) peptide.

### DETAILED DESCRIPTION OF THE INVENTION

The delivery of natriuretic peptides stabilized in an aqueous solution is disclosed. Stabilized aqueous solutions of natriuretic peptides with a drug provisioning component that can include both programmable and constant rate subcutaneous infusion pumps, implanted or percutaneous vascular access ports, direct delivery catheter systems, local drug-release devices or any other type of medical device that can be adapted to deliver a therapeutic to a patient are also disclosed. The drug provisioning component can administer the natriuretic peptide subcutaneously, intramuscularly, or intravenously or direct to the kidney at a fixed, pulsed, continuous or variable rate. One embodiment of the invention contemplates subcutaneous delivery using an infusion pump at a continuous rate to maintain a specified plasma concentration of the natriuretic peptides. Natriuretic peptides and their sequences are disclosed in U.S. Pat. No. 5,691,310 and U.S. Patent App. Pub. Nos. 2006/0205642, 2008/0039394, 2009/0062206, and 2009/20170196, each of which is incorporated by reference herein in its entirety.

### DEFINITIONS

Unless defined otherwise, all technical and scientific terms used herein generally have the same meaning as commonly understood by one of ordinary skill in the relevant art. Generally, the nomenclature used herein for drug delivery, pharmacokinetics, pharmacodynamics, and peptide chemistry is well known and commonly employed in the art. Further, the techniques for the discussed procedures are generally performed according to conventional methods in the art.

The articles “a” and “an” are used herein to refer to one or to more than one (i.e., to at least one) of the grammatical object of the article. By way of example, “an element” means one element or more than one element.

The terms “administering,” “administer,” “delivering,” “deliver,” “introducing,”, and “introduce” can be used interchangeably to indicate the introduction of a therapeutic composition or agent into the body of a patient, including a natriuretic peptide. The therapeutic composition or agent can be introduced through any means including intravenous infusion and subcutaneous infusion.

The term “comprising” includes, but is not limited to, whatever follows the word “comprising.” Thus, use of the term indicates that the listed elements are required or mandatory but that other elements are optional and may or may not be present.

The term “consisting of” includes and is limited to whatever follows the phrase the phrase “consisting of.” Thus, the phrase indicates that the limited elements are required or mandatory and that no other elements may be present.

The phrase “consisting essentially of” includes any elements listed after the phrase and is limited to other elements that do not interfere with or contribute to the activity or action specified in the disclosure for the listed elements. Thus, the phrase indicates that the listed elements are required or mandatory but that other elements are optional and may or may not be present, depending upon whether or not they affect the activity or action of the listed elements.

“Chronic Kidney Disease” (CKD) is a condition characterized by the slow loss of kidney function over time. The most common causes of CKD are high blood pressure, diabetes, heart disease, and diseases that cause inflammation in the kidneys. Chronic kidney disease can also be caused by infections or urinary blockages. If CKD progresses, it can lead to end-stage renal disease (ESRD), where the kidneys fail to function at a sufficient level.

“Pharmaceutically acceptable” is meant to encompass any carrier, which does not interfere with effectiveness of the biological activity of the active ingredient and that is not toxic to the host to which it is administered.

The term “inert gas” refers to any gas that one having ordinary skill in the art will recognize as not readily undergoing chemical reactions including oxidation reactions. Inert gases include nitrogen, helium, argon and noble gases.

“Drug provisioning component” or “drug provisioning apparatus” encompasses any and all devices that administers a therapeutic agent to a patient and includes infusion pumps, implanted or percutaneous vascular access ports, direct delivery catheter systems, local drug-release devices or any other type of medical device that can be adapted to deliver a therapeutic to a patient. The drug provisioning component and the control unit may be “co-located,” which means that these two components, in combination, may make up one larger, unified unit of a system.

The term “percent recovery” refers to the mass of proteins, peptides or polypeptides in a solution expressed as a percent relative to the mass of proteins, peptides or polypeptides in an initial or starting solution before exposure of the solution to elevated temperature or mechanical stress.

The term “stability” refers to the degree of recovery or purity of a protein, peptide or polypeptide from a solution and/or the maintenance of the purity of the protein, peptide or polypeptide in solution.

“Glomerular filtration rate” describes the flow rate of filtered fluid through the kidney. The estimated glomerular filtration rate or “eGFR” is a measure of filtered fluid based on a creatinine test and calculating the eGFR based on the results of the creatinine test.

The term “initial composition,” “initial therapeutic composition,” “starting composition,” or “starting therapeutic composition” refers to a composition having one or more active agents, such as a natriuretic peptide, that is newly constituted and has not been stored for a significant period of time.

A “patch pump” is a device that adheres to the skin, contains a medication, and can deliver the drug over a period of time, either transdermally, iontophoretically, or via an integrated or separate subcutaneous mini-catheter.

The term “delivering,” “deliver,” “administering,” and “administers” can be used interchangeably to indicate the introduction of a therapeutic or diagnostic agent into the body of a patient in need thereof to treat a disease or condition, and can further mean the introduction of any agent into the body for any purpose.

The term “therapeutically effective amount” refers to an amount of an agent (e.g., natriuretic peptides) effective to treat at least one symptom of a disease or disorder in a patient. The “therapeutically effective amount” of the agent for administration may vary based upon the desired activity, the diseased state of the patient being treated, the dosage form, method of administration, patient factors such as the patient's sex, genotype, weight and age, the underlying causes of the condition or disease to be treated, the route of administration and bioavailability, the persistence of the administered agent in the body, evidence of natriuresis and/or diuresis, the type of formulation, and the potency of the agent.

The term “treating” and/or “treatment” refers to refer to the management and care of a patient having a pathology or condition by administration of one or more therapies and/or therapeutic compositions contemplated by the present invention. Treating also includes administering one or more methods or therapeutic compositions of the present invention or using any of the systems, devices or compositions of the present invention in the treatment of a patient. As used herein, “treatment” or “therapy” refers to both therapeutic treatment and prophylactic or preventative measures. “Treating” or “treatment” does not require complete alleviation of signs or symptoms, does not require a cure, and includes protocols having only a marginal or incomplete effect on a patient.

A “subject” or “patient” is a member of any animal species, preferably a mammalian species, optionally a human. The subject can be an apparently healthy individual, an individual suffering from a disease, or an individual being treated for a disease.

The term “sample” refers to a quantity of a biological substance that is to be tested for the presence or absence of one or more molecules.

“Absorption” refers to the transition of drug from the site of administration to the blood circulation.

“Adsorption” refers to the interaction of a substance with a surface where the substance adheres to the surface.

The “distal tip” of a catheter is the end that is situated farthest from a point of attachment or origin, and the end closest to the point of attachment or origin is known as the “proximal” end.

A “direct delivery catheter system,” as used herein is a catheter system for guiding an elongated medical device into an internal bodily target site. The system can have a distal locator for locating a target site prior to deployment of the catheter. The catheter can be introduced through a small incision into the bodily vessel, channel, canal, or chamber in question; or into a bodily vessel, channel, canal, or chamber that is otherwise connected to the site of interest (or target site), and then guided through that vessel to the target site.

The term “headspace” refers to the area of a container that is occupied by gas and not occupied by a liquid.

The term “inert gas” refers to any gas that one having ordinary skill in the art will recognize as not readily undergoing chemical reactions including oxidation reactions. Inert gases include nitrogen, helium, argon and noble gases.

The terms “protein,” “peptide,” and “polypeptide” as used herein, describes an oligopeptide, polypeptide, or peptide polymer in which the monomers are amino acids that are joined together through amide bonds in at least part of the molecule. The present invention also embraces recombination peptides such as recombinant human ANP (hANP) obtained from bacterial cells after expression inside the cells. It will be understood by those of skill in the art that the peptides and recombinant peptides of the present invention can be made by varied methods of manufacture wherein the peptides of the invention are not limited to products of any of the specific exemplary processes listed herein.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. The present invention also provides for analogs of proteins or peptides which comprise a protein as identified above.

The term “fragment,” as used herein, refers to a polypeptide that comprises at least six contiguous amino acids of a polypeptide from which the fragment is derived. In preferred embodiments, a fragment refers to a polypeptide that comprises at least 10 contiguous amino acids of a polypeptide from which the fragment is derived, more preferably at least 10 contiguous amino acids, still more preferably at least 15 contiguous amino acids, and still more preferably at least 20 contiguous amino acids of a polypeptide from which the fragment is derived.

The term “natriuretic peptide fragment” refers to a fragment of any natriuretic peptide defined and described herein.

As used herein, “cardiovascular disease” refers to various clinical diseases, disorders or conditions involving the heart, blood vessels, or circulation. Cardiovascular disease includes, but is not limited to, coronary artery disease, peripheral vascular disease, hypertension, myocardial infarction, and heart failure.

As used herein, “heart failure” (HF) refers to a condition in which the heart cannot pump blood efficiently to the rest of the body. Heart failure may be caused by damage to the heart or narrowing of the arteries due to infarction, cardiomyopathy, hypertension, coronary artery disease, valve disease, birth defects or infection. Heart failure may also be further described as chronic, congestive, acute, decompensated, systolic, or diastolic. The NYHA classification describes the severity of the disease based on functional capacity of the patient and is incorporated herein by reference.

Relating to heart failure, for example, “increased severity” of cardiovascular disease refers to the worsening of the disease as indicated by increased New York Heart Association (NYHA) classification, and “reduced severity” of cardiovascular disease refers to an improvement of the disease as indicated by reduced NYHA classification.

The “renal system,” as defined herein, comprises all the organs involved in the formation and release of urine including the kidneys, ureters, bladder and urethra.

The term “phosphate buffer” refers to a buffer that contains monohydrogen phosphate ions (HPO₄²⁻) and dihydrogen phosphate ions (H₂PO₄⁻) regardless of the source from which such ions originate.

“Proteinuria,” is a condition in which urine contains an abnormal amount of protein. One form of proteinuria is albuminuria, where the urine contains an abnormal amount of albumin protein. Albumin is the main protein in the blood. Healthy kidneys filter out waste products while retaining necessary proteins such as albumin Most proteins are too large to pass through the glomeruli into the urine. However, proteins from the blood can leak into the urine when the glomeruli of the kidney are damaged. Hence, proteinuria is one indication of chronic kidney disease (CKD).

“Chronic kidney disease” (CKD) is a condition characterized by the slow loss of kidney function over time. The most common causes of CKD are high blood pressure, diabetes, heart disease, and diseases that cause inflammation in the kidneys. Chronic kidney disease can also be caused by infections or urinary blockages. If CKD progresses, it can lead to end-stage renal disease (ESRD), where the kidneys fail completely. In the Cardiorenal Syndrome (CRS) classification system, CRS Type I (Acute Cardiorenal Syndrome) is defined as an abrupt worsening of cardiac function leading to acute kidney injury; CRS Type II (Chronic Cardiorenal syndrome) is defined as chronic abnormalities in cardiac function (e.g., chronic congestive heart failure) causing progressive and permanent chronic kidney disease; CRS Type III (Acute Renocardiac Syndrome) is defined as an abrupt worsening of renal function (e.g., acute kidney ischaemia or glomerulonephritis) causing acute cardiac disorders (e.g., heart failure, arrhythmia, ischemia); CRS Type IV (Chronic Renocardiac syndrome) is defined as chronic kidney disease (e.g., chronic glomerular disease) contributing to decreased cardiac function, cardiac hypertrophy and/or increased risk of adverse cardiovascular events; and CRS Type V (Secondary Cardiorenal Syndrome) is defined as a systemic condition (e.g., diabetes mellitus, sepsis) causing both cardiac and renal dysfunction (Ronco et al., Cardiorenal syndrome, J. Am. Coll. Cardiol. 2008; 52:1527-39). It is understood that CKD, as defined in the present invention, contemplates CKD regardless of the direction of the pathophysiological mechanisms causing CKD and includes CRS Type II and Type IV among others.

“Hemodynamics” is the study of blood flow or circulation. The factors influencing hemodynamics are complex and extensive but include cardiac output (CO), circulating fluid volume, respiration, vascular diameter and resistance, and blood viscosity. Each of these may in turn be influenced by physiological factors. Hemodynamics depends on measuring the blood flow at different points in the circulation. Blood pressure is the most common clinical measure of circulation and provides a peak systolic pressure and a diastolic pressure. “Blood pressure” (BP) is the pressure exerted by circulating blood upon the walls of blood vessels.

The term “intrinsic” is used herein to describe something that is situated within or belonging solely to the organ or body part on which it acts. Therefore, “intrinsic natriuretic peptide generation” refers to a subject's making or releasing of one or more natriuretic peptides by its respective organ(s).

“Cardiac output” (CO), or (Q), is the volume of blood pumped by the heart per minute (mL/min) Cardiac output is a function of heart rate and stroke volume. The heart rate is simply the number of heart beats per minute. The stroke volume is the volume of blood, in milliliters (mL), pumped out of the heart with each beat. Increasing either heart rate or stroke volume increases cardiac output. Cardiac Output in mL/min=heart rate (beats/min)×stroke volume (mL/beat).

A “buffer,” “buffer composition,” or “buffer solution” is an aqueous solution consisting of a mixture of a weak acid and its conjugate base or a weak base and its conjugate acid. A buffer solution can be formed by adding a weak acid to a solution wherein a portion of the weak acid spontaneously forms its conjugate base by hydrolysis or wherein the conjugate base forms by titration with a base. Similarly, a buffer solution can be formed by adding a weak base to a solution wherein a portion of the weak base spontaneously forms its conjugate acid by hydrolysis or wherein the conjugate acid forms by titration with an acid. A buffer solution may contain more than one species of a weak acid and its conjugate base or a weak base and its conjugate acid. Buffer solutions include solutions containing a weak acid or the conjugate acid of a weak base having a pKₐ from about 5 to about 8. A buffer solution can have pH within about 1.5 units from the pKa of a weak acid or conjugate acid of a weak base present in the buffer solution. Buffer solutions include solutions containing tris(hydroxymethyl)methylamine, monobasic phosphate, dibasic phosphate or phosphoric acid. A buffer solution does not require a specific concentration of a weak acid its conjugate base or a weak base and its conjugate acid.

The term “peptide chain” refers to the part of a molecule formed from a region of peptide bonds between amino acid resides, where the peptide chain can be covalently linked to another peptide chain through the side-chains of the amino acid residues, such as a disulfide bridge.

The term “recovery” in relation to the presence of proteins, peptides or polypeptides in a solution refers to a percentage of the total mass of proteins, peptides or polypeptides present in the solution at a beginning of time period compared to the total mass of proteins, peptides or polypeptides present in the same solution after the elapse of a time period.

The term “percent recovery” refers to the mass of a proteins, peptides or polypeptides in a solution expressed as a percent relative to the mass of proteins, peptides or polypeptides in an initial or starting solution before exposure of the solution to elevated temperature or mechanical stress or an elapse of time.

The term “purity” refers to percentage of mass of a protein, peptide or polypeptide in a solution that has the same chemical identity. Chemical identity can be determined by suitable analytical techniques such as high performance liquid chromatography and reverse-phase high performance liquid chromatography.

The term “change in relative purity” refers to a normalized change in purity, as measured as a percentage, for a protein, peptide or polypeptide from an initial purity to a purity measured at a later time.

The terms “natriuretic” or “natriuresis” refer to the ability of a substance to increase sodium clearance from a subject.

The terms “renal protective” and “reno-protective” refer to the ability of a substance to improve one or more functions of the kidneys of a subject, including natriuresis, diuresis, cardiac output, hemodynamics or glomerular filtration rate, or to lower the blood pressure of the subject.

The term “at a temperature” or any reference to maintain any mixture, solution or composition refer to the maintenance of the mixture, solution or composition at the specified temperature for at least a majority of a referenced time period, where the mixture, solution or composition can be at a different temperature for a portion of the time period.

The term “high degree of stability” refers to the ability of a therapeutic composition to maintain a substantially unchanged chemical makeup over a stated period of time, including the chemical identity and concentration of a natriuretic peptide species present in the therapeutic composition. Due to the substantially unchanged chemical makeup of the therapeutic composition after an elapse of time, administration of a unit volume of the therapeutic composition to a subject or patient delivers substantially the same amount of the natriuretic peptide species to the patient over the stated period of time. The therapeutic composition is substantially unchanged in chemical makeup when administration of the therapeutic composition to a patient or subject results in an area under the curve (AUC) for the natriuretic peptide species within 80% to 125% as that for the starting therapeutic composition. The therapeutic composition can further be described as having a high degree of stability under conditions of elevated temperatures and/or mechanical stress.

The phrase “area under the curve” or “AUC” refers to the area under a plasma concentration versus time curve. It indicates a measurement of drug absorption. AUC is described by the following formula:

AUC=∫₀^{∞}C(t)dt

where C(t) indicates the concentration of the drug in the plasma at time t.

### Natriuretic Peptides

Chronic kidney disease (CKD), also known as chronic renal disease, is a progressive loss in renal function over a period of months or years. CKD is a major U.S. public health concern with recent estimates suggesting that more than 26 million adults in the U.S. have the disease. Heart failure (HF) is a related condition in which the heart's ability to pump blood through the body is impaired. If left untreated, compensated HF can deteriorate to a point where a person undergoes acute decompensated heart failure (ADHF), which is the functional deterioration of HF. One pharmaceutical approach to treat ADHF is the use of Nesiritide (B-type natriuretic peptide), which is an FDA approved therapeutic option that lowers elevated filing pressures and improves dyspnea. Nesiritide is the recombinant form of the 32 amino acid human B-type natriuretic peptide, which is normally produced by the ventricular myocardium; SPKMVQGSGCFGRKMDRISSSSGLGCKVLRRH (SEQ ID No. 1). The drug facilitates cardiovascular fluid homeostasis through counter-regulation of the renin-angiotensin-aldosterone system and promotion of vasodilation, natriuresis, and diuresis. Nesiritide is administered intravenously usually by bolus injection, followed by IV infusion. Another approved atrial natriuretic type peptide is human recombinant atrial natriuretic peptide (ANP), SLRRSSCFGGRMDRIGAQSGLGCNSFRY (SEQ ID No. 2), Carperitide, which has been approved for the clinical management of ADHF in Japan since 1995. Carperitide is also administered via intravenous infusion. Another peptide under study is human recombinant urodilatin (URO), Ularitide.

The natriuretic peptides have been the focus of intense study subsequent to the seminal work by DeBold et al. on the potent diuretic and natriuretic properties of atrial extracts and its precursors in atrial tissues (A rapid and potent natriuretic response to intravenous injection of atrial myocardial extract in rats, Life Sci., 1981; 28(1): 89-94). Some natriuretic peptides are a family of peptides having a 17 amino acid disulphide ring structure acting in the body to oppose the activity of the renin-angiotensin system. That is, a natriuretic peptide may have an intramolecular disulfide bond between two cysteine residues in the same peptide chain, wherein 15 amino acid residues in the peptide chain are located between the two cysteine residues forming the disulfide. In humans, the family consists of atrial natriuretic peptide (ANP), brain natriuretic peptide (BNP) of myocardial cell origin, C-type natriuretic peptide (CNP) of endothelial origin, and urodilatin (URO), which is thought to be derived from the kidney. Atrial natriuretic peptide (ANP), alternatively referred to in the art as Atrial natriuretic factor (ANF), is secreted by atrial myocytes in response to increased intravascular volume. Once ANP is in the circulation, its effects are primarily on the kidney, vascular tissue, and adrenal gland. ANP leads to the excretion of sodium and water by the kidneys and to a decrease in intravascular volume and blood pressure. Brain natriuretic peptide (BNP) also originates from myocardial cells and circulates in human plasma similar to ANP. BNP is natriuretic, renin inhibiting, vasodilating, and lusitropic. C-type natriuretic peptide (CNP) is of endothelial cell origin and functions as a vasodilating and growth-inhibiting polypeptide. Natriuretic peptides have also been isolated from a range of other vertebrates. For example, Dendroaspis angusticeps natriuretic peptide is detected in the venom of Dendroaspis angusticeps (the green mamba); CNP analogues are cloned from the venom glands of snakes of the Crotalinae subfamily; Pseudocerastes persicus natriuretic peptide is isolated from the venom of the Iranian snake (Pseudocerastes persicus), and three natriuretic-like peptides (TNP-a, TNP-b, and TNP-c) are isolated from the venom of the Inland Taipan (Oxyuranus microlepidotus). Because of the capacity of natriuretic peptides to restore hemodynamic balance and fluid homeostasis, they can be used to manage cardiopulmonary and renal symptoms of cardiac disease due to their vasodilator, natriuretic and diuretic properties.

The five major ANP derived hormones are atrial long-acting natriuretic peptide (LANP), kaliuretic peptide (KP), urodilatin (URO), atrial natriuretic peptide (ANP), and vessel dilator (VD). These hormones function via well-characterized receptors located on the cell surface linked to a guanylyl cyclase enzyme to generate an intracellular signal, and have significant blood pressure lowering, diuretic, sodium and/or potassium excreting properties in healthy humans. In particular, ANP is a biological hormone, also referred to as atrial natriuretic factor (ANF), which has been implicated in diseases and disorders involving volume regulation, such as congestive heart failure, hypertension, liver disease, nephrotic syndrome, and acute and chronic renal failure. In the heart, ANP has growth regulatory properties in blood vessels that inhibit smooth muscle cell proliferation (hyperplasia) as well as smooth muscle cell growth (hypertrophy). ANP also has growth regulatory properties in a variety of other tissues, including brain, bone, myocytes, red blood cell precursors, and endothelial cells. In the kidneys, ANP causes antimitogenic and antiproliferative effects in glomerular mesangial cells. ANP has been infused intravenously to treat hypertension, heart disease, acute renal failure and edema, and shown to increase the glomerular filtration rate (GFR) and filtration fraction. ANP has further been shown to reduce proximal tubule sodium ion concentration and water reabsorption, inhibit net sodium ion reabsorption and water reabsorption in the collecting duct, lower plasma renin concentration, and inhibit aldosterone secretion. Further, administration of ANP has resulted in mean arterial pressure reduction.

Within the 126 a.a. ANP prohormone are four peptide hormones: long acting natriuretic peptide (LANP) (also known as proANP 1-30) (a.a. 1-30), vessel dilator (a.a. 31-67), kaliuretic peptide (a.a. 79-89), and atrial natriuretic peptide (a.a. 99-126), whose main known biologic properties are blood pressure regulation and maintenance of plasma volume in animals and humans. The fifth member of the atrial natriuretic peptide family, urodilatin (URO) (ANP a.a. 95-126) is isolated from human urine and has an N-terminal extension of four additional amino acids, as compared with the circulating form of ANP (a.a. 99-126). This hormone is synthesized in the kidney and exerts potent paracrine renal effects (Meyer, M. et al., Urinary and plasma urodilatin mearured by a direct RIA using a highly specific antiserum, Clin. Chem., 1998; 44(12):2524-2529). Several studies have suggested that URO is involved in the physiological regulation of renal function, particularly in the control of renal sodium and water excretion wherein a concomitant increase in sodium and URO excretion was observed after acute volume load and after dilation of the left atrium. Additionally, infusions and bolus injections of URO in rats and healthy volunteers have also revealed the pharmacological potency of this natriuretic peptide wherein intense diuresis and natriuresis as well as a slight reduction in blood pressure are the most prominent effects. The strength and duration of these effects differ considerably from ANP a.a. 99-126.

The peptide sequences for the four ANP peptide hormones, long acting natriuretic peptide (LANP) (also known as proANP 1-30) (a.a. 1-30), vessel dilator (VD) (a.a. 31-67), kaliuretic peptide (KP) (a.a. 79-89), and atrial natriuretic peptide (ANP) (a.a. 99-126), are as follows:

proANP or LANP, (a.a. 1-30)

(SEQ ID No. 3)

NPMYNAVSNADLMDFKNLLDHLEEKMPLED
Vessel Dilator, (a.a. 31-67)

(SEQ ID No. 4)

EVVPPQVLSEPNEEAGAALSPLPEVPPWTGEVSPAQR
Kaliuretic Peptide, (a.a. 79-98)

(SEQ ID No. 5)

SSDRSALLKSKLRALLTAPR
ANP, (a.a. 99-126)

(SEQ ID No. 6)

SLRRSSCFGGRMDRIGAQSGLGCNSFRY

The fifth member of the atrial natriuretic peptide family, urodilatin (URO) (ANP a.a. 95-126) is isolated from human urine and has an N-terminal extension of four additional amino acids, as compared with the circulating form of ANP (a.a. 99-126). This hormone is synthesized in the kidney and exerts potent paracrine renal effects. (Meyer, M. et al., Urinary and plasma urodilatin measured by a direct RIA using a highly specific antiserum, Clin. Chem., 1998; 44(12):2524-2529). Several studies have suggested that URO is involved in the physiological regulation of renal function, particularly in the control of renal sodium and water excretion wherein a concomitant increase in sodium and URO excretion was observed after acute volume load and after dilation of the left atrium. Additionally, infusions and bolus injections of URO in rats and healthy volunteers have also revealed the pharmacological potency of this natriuretic peptide wherein intense diuresis and natriuresis as well as a slight reduction in blood pressure are the most prominent effects. The strength and duration of these effects differ considerably from ANP a.a. 99-126. The sequence for urodilatin is provided in SEQ ID No. 7.

### Urodilatin (a.a. 95-126)

### (SEQ ID No. 7) TAPRSLRRSSCFGGRMDRIGAQSGLGCNSFRY

Of the 20 amino acids commonly forming peptides and proteins, valine, isoleucine, leucine, methionine, proline, phenylalanine, and tryptophan are particularly hydrophobic. In certain embodiments, a protein, peptide or polypeptide contained in a therapeutic composition has about 45% of the amino acid resides therein selected from valine, isoleucine, leucine, methionine, proline, phenylalanine, and tryptophan. In certain other embodiments, a protein, peptide or polypeptide contained in a therapeutic composition has about 40% of the amino acid resides therein selected from valine, isoleucine, leucine, methionine, proline, phenylalanine, and tryptophan. In certain embodiments, the protein, peptide or polypeptide in the therapeutic protein composition has from about 20 to about 40 amino acid residues.

The peptides described herein, can be synthesized using solid phase methods on an ABI 431A Peptide Synthesizer (PE Biosystems, Foster City, Calif.) on a pre-loaded Wang resin with N-Fmoc-L-amino acids (SynPep, Dublin, Calif.). The synthesized peptide can then be confirmed using high-performance liquid chromatography or mass spectrometry, such as by electrospray ionization mass analysis on a Perkin/Elmer Sciex API 165 Mass Spectrometer (PE Biosystems).

As discussed above, many peptides synthesized by using solid phase methods can have limited tertiary structure and thereby have limited protection from oxidation, hydrolysis, proteolyic attack, aggregation or other structural changes when formulated in aqueous compositions. It is desirable for formulations of therapeutic agents, including formulations of synthetic peptides, to have a high degree of stability over time. Stability is the tendency of the chemical composition and physical properties of the therapeutic formulation to remain unchanged over time. Stable formulations are indicated by a consistent recovery of peptide or protein mass from solution, which is an indication of a lack of surface adsorption of the peptide and/or a lack of aggregation of the peptide that results in precipitation. Stable formulations are also indicated by a lack of chemical change to the one or more peptides or proteins in the therapeutic composition. Peptides, particularly peptides synthesized by solid phase methods, are discrete molecular species having uniform molecular weight with the exception of ionizable groups. Chemical modifications to a peptide include hydrolysis of the peptide backbone to form two or more peptides and/or modifications to side-chains such as oxidation, esterification, etc. Chemical modifications to a peptide do not necessarily cause the removal of mass from the aqueous formulation. However, chemical modifications to a peptide affect the stability of therapeutic formulations since the peptide species having pharmaceutical properties is degraded by the degree of chemical change.

A stable therapeutic composition has a near constant peptide content or recovery over time and exhibits consistency in the molecular species or purity observed to be present in the composition. For example, a therapeutic composition formulated with one molecular peptide species will contain substantially only that particularly molecular peptide species over time. Likewise, a therapeutic composition formulated with two molecular peptide species will contain substantially only those two species in the same proportion over time. It should be noted that the observation of a high degree of recovery from a therapeutic composition or purity of molecular species in a therapeutic composition is an indication of overall stability.

In any embodiment, at least about 80% of the mass and higher of one or more peptides contained in a therapeutic composition are recoverable and still distributed in the composition after storage for 14 days and the purity of such recovered peptides is at least about 90%. In any embodiment, at least about 85% of the mass and higher of one or more peptides contained in a therapeutic composition are recoverable and still distributed in the composition after storage for 14 days and the purity of such recovered peptides is at least about 90%. In certain other embodiments, at least about 92% of the mass of one or more peptides contained in a therapeutic composition are recoverable and still distributed in the composition after storage for 14 days and the purity of such recovered peptides is at least about 92%. In certain additional embodiments, at least about 95% of the mass of one or more peptides contained in a therapeutic composition are recoverable and still distributed in the composition after storage for 14 days and the purity of such recovered peptides is at least about 95%. In still further embodiments, at least about 97% of the mass of one or more peptides contained in a therapeutic composition are recoverable and still distributed in the composition after storage for 14 days and the purity of such recovered peptides is at least about 97%. In yet further embodiments, at least about 98% of the mass of one or more peptides contained in a therapeutic composition are recoverable and still distributed in the composition after storage for 14 days and the purity of such recovered peptides is at least about 98%.

As used herein, a composition having a high degree of stability maintains a consistent deliverable amount of an active agent or peptide over a period of time. That is, a composition has a high degree of stability when a particular dosing regimen of the composition results in substantially the same amount of the active agent or peptide being present in the plasma of a subject or patient after storage of the composition for a period of time and/or exposure of the composition to heat and/or mechanical stress. As such, a composition having a high degree of stability during storage for a stated time period and under specified conditions results in a consistent AUC upon administration to a patient compared with the starting therapeutic composition. For example, the AUC after storage can be from 80 to 125% of the AUC that results from administration of the initial therapeutic composition.

In certain embodiments, a therapeutic composition has a high degree of stability for a period of at least 14 days. In certain other embodiments, a therapeutic composition has a high degree of stability for a period of at least 6 days. In other embodiments, a therapeutic composition has a high degree of stability for a period of at least 14 days. In certain embodiments, a therapeutic composition has a high degree of stability when stored at a temperature from 25 to about 45° C. for a period of at least 14 days. In certain other embodiments, a therapeutic composition has a high degree of stability when stored at a temperature from 25 to about 45° C. for a period of at least 6 days. In other embodiments, a therapeutic composition has a high degree of stability when stored at a temperature from 25 to about 45° C. for a period of at least 4 days. In certain embodiments, a therapeutic composition has a high degree of stability when stored at a temperature from about 4 to about 15° C. for a period of at least 4, 6 or 14 days.

One obstacle to delivering peptides in a clinically effective manner is that peptides generally have poor delivery properties due to the presence of endogenous proteolytic enzymes, which are able to quickly metabolize many peptides at most routes of administration. Further, peptides may decompose and/or absorb on the surface of a container during storage or onto the surfaces of the conduits, W lines, and pumps used to deliver peptides either by bolus or infusion to a patient via an intravenous or subcutaneous (SQ) administration route. Such complications are amplified for therapeutic protein compositions formulated for delivery by a continuous infusion device or pump, where the therapeutic protein composition will experience elevated temperatures and mechanical stress.

In certain embodiments, the composition of the therapeutic protein has a high degree of purity after being administered from a provisioning apparatus over an extended period of time, for example after a 6-day period of time. In certain embodiments, at least about 90% of the mass of one or more peptides contained in a therapeutic composition are present in a volume of the therapeutic composition administered by a provisioning apparatus over a 6-day period and the purity of such administered peptides is at least about 90%. In certain further embodiments, at least about 95% of the mass of one or more peptides contained in a therapeutic composition are present in a volume of the therapeutic composition administered by a provisioning apparatus over a 6-day period and the purity of such administered peptides is at least about 95%. In certain additional embodiments, at least about 97% of the mass of one or more peptides contained in a therapeutic composition are present in a volume of the therapeutic composition administered by a provisioning apparatus over a 6-day period and the purity of such administered peptides is at least about 97%. In still further embodiments, at least about 98% of the mass of one or more peptides contained in a therapeutic composition are present in a volume of the therapeutic composition administered by a provisioning apparatus over a 6-day period and the purity of such administered peptides is at least about 98%.

In any embodiment, at least about 80% of the mass of one or more peptides contained in a therapeutic composition is present in a volume of the therapeutic composition administered by a provisioning apparatus after a period of time and the purity of such administered peptides is at least about 80%. In any embodiment, at least about 85% of the mass of one or more peptides contained in a therapeutic composition is present in a volume of the therapeutic composition administered by a provisioning apparatus after a period of time and the purity of such administered peptides is at least about 85%.

Those skilled in the art will readily understand that methods for synthesizing artificial peptides may not be capable of producing a peptide product having 100% purity. That is, a peptide produced by a method such as solid phase synthesis will contain impurities undermost conditions such that a composition formed from the synthesized peptide will have a purity less than 100%. Peptides produced by recombinant methods will also have purities less than 100% in most instances.

As such, an initial formulation a composition containing a peptide will have a starting purity of less than 100%. A change in relative purity of the peptide can be measured from the initial purity of the peptide over a period of time. The initial purity of a peptide is the purity of the peptide as synthesized or otherwise obtained and a measured purity is the purity of a composition containing the peptide after a period of time. The change in relative purity can be calculated using the following equation:

$$Change in Relative Purity = \frac{Initial Purity - Measured Purity}{Initial Purity}$$

For example, if a peptide has an initial purity of 98% and a measured purity in a composition of 95% after 1 week, then the change in purity is 3% while the change in relative purity is 3.06%.

### Stability of Therapeutic Protein Compositions

The therapeutic proteins, peptides or polypeptides, including VD and KP, have enhanced stability in buffers containing tris-(hydroxymethyl)-aminomethane (“Tris”) or phosphate buffers. Aqueous compositions of therapeutic proteins or peptides are typically formulated several weeks, if not months, prior to actually use for administration to a patient. Further, the stability of a therapeutic protein composition can be affected based upon the type of container holding the therapeutic protein composition. For example, a therapeutic protein can be distributed to commercial pharmacies in a glass container. However, when used in a pump or infusion device, the therapeutic protein composition can come into contact with plastic or metal surfaces that can affect the stability of any proteins, peptides or polypeptides contained in the therapeutic composition.

During use of the therapeutic composition in an infusion device or pump, the composition is exposed to elevated temperature in addition to mechanical stress. Elevated temperature is the result of both location of the pump near the body heat of the subject. In some embodiments, a therapeutic composition is delivered to a patient having a temperature that is not substantially different from the body temperature of an individual. The therapeutic protein compositions disclosed herein have enhanced stability in the temperature range from about 25 to about 45° C. and any range in between. The therapeutic protein compositions also have enhanced stability at room temperatures of about 20 to about 30° C. and any range in between. For long term storage, the therapeutic protein compositions described herein are stable for storage at refrigerated temperatures from about 4 to about 15° C. and any range in between.

The therapeutic protein compositions described herein have a pH from about 6.5 to about 7.6 and any range in between. The therapeutic protein composition can have a pH from about 6.5 to 7.6 at any temperature or have a composition such that the pH is from about 6.5 to 7.6 when the therapeutic protein composition is adjusted to a temperature of 25° C.

In certain embodiments, the therapeutic protein composition contains additional components. Examples of additional components include meta-cresol (m-cresol) and glycerol. In certain embodiments, the concentration of m-cresol in the therapeutic protein composition is from about 0.15 to about 0.315% by weight, including all possible sub-ranges, such as from 0.15-0.2%, from 0.15-0.25%, from 0.15-0.3%, from 0.15-0.31%, from 0.2-0.25%, from 0.2-0.3%, from 0.2-0.31%, from 0.2-0.315%, from 0.215%-0.23%, from 0.215%-0.235%, from 0.215%-0.27%, from 0.215%-0.3%, from 0.215%-0.315%, from 0.23%-0.24%, from 0.23%-0.245%, from 0.23%-0.25%, from 0.23%-0.26%, from 0.23%-0.27%, etc. In certain embodiments, the concentration of glycerol in the therapeutic protein composition ranges from greater than 0 to about 5%, as represented by the range from n to (n+i), where n={xε [symbol] |0<x≦5} and i={yε [symbol] |0≦y≦(5−n)}. In certain other embodiments, the concentration of glycerol in the therapeutic protein composition is from about 0.1 to about 5% by weight.

In any embodiment, the concentration of Tris buffer in the therapeutic protein composition is from about 5 to about 200 mM including all possible sub-ranges, for example from about 5 to about 100 mM, 10 to about 70 mM, 10 to about 70 mM, 20 to about 65 mM, 25 to about 50 mM, 30 to about 70 mM, 40 to about 60 mM, 50 to about 70 mM, 45 to about 55 mM, 9 to about 63 mM, 14 to about 56 mM, 27 to about 50 mM, 35 to about 68 mM, 11 to about 47 mM, 34 to about 66 mM, 29 to about 57 mM, 22 to about 68 mM, or 50 to about 70 mM.

In any embodiment, the concentration of phosphate buffer (dihydrogen phosphate salts and monohydrogen phosphate salts, combined) in the therapeutic protein composition is from about 0.2 to about 10 grams per liter including all possible sub-ranges. For example, the concentration of phosphate buffer in the therapeutic protein composition is from about 0.5 to about 5 grams per liter or from about 1 to about 4 grams per liter, from about 2 to about 15 grams per liter, or from about 5 to about 10 grams per liter. In further embodiments, the therapeutic protein composition contains a physiological amount of sodium chloride.

In any embodiment, the therapeutic protein composition has a concentration of one or more proteins, polypeptides and peptides from about 0.05 to about 20 mg/mL including all possible sub-ranges, such as from about 0.10 to about 15 mg/mL, 0.05 to about 10 mg/mL, 0.10 to about 7 mg/mL, 0.10 to about 5 mg/mL, 3 to about 7 mg/mL, 4 to about 8 mg/mL, 2 to about 4 mg/mL, 3 to about 9 mg/mL, 6 to about 10 mg/mL, or from about 0.05 to about 8 mg/mL. In other embodiments, the therapeutic protein composition can be diluted by a factor from about 10 to about 100 prior to administration to a subject.

In any embodiment, the recovery of a natriuretic peptide from a solution stored and administered from a provisioning apparatus is about 80% or more when the provisioning apparatus is operated at a temperature from about 25 to about 45° C. for a period of about 4 days or more. In certain other embodiments, the recovery of a natriuretic peptide from a solution stored and administered from a provisioning apparatus is about 97% or more when the provisioning apparatus is operated at a temperature from about 25 to about 45° C. for a period of about 4 days or more. In certain embodiments, the purity of a natriuretic peptide recovered from a solution stored and administered from a provisioning apparatus is about 92% or more when the provisioning apparatus is operated at a temperature from about 25 to about 45° C. for a period of about 4 days or more. In certain other embodiments, the purity of a natriuretic peptide recovered from a solution is about 95% or more when the provisioning apparatus is operated at a temperature from about 25 to about 45° C. for a period of about 4 days or more. In certain additional other embodiments, the purity of a natriuretic peptide recovered from a solution stored and administered from a provisioning apparatus is about 97% or more when the provisioning apparatus is operated at a temperature from about 25 to about 45° C. for a period of about 4 days or more. In certain other embodiments, the purity of a natriuretic peptide recovered from a solution stored and administered from a provisioning apparatus is about 98% or more when the provisioning apparatus is operated at a temperature from about 25 to about 45° C. for a period of about 4 days or more.

In any embodiment, the recovery of a natriuretic peptide from a solution stored in a container is about 92% when stored at a temperature from about 4 to about 15° C. for a period of about 4 days or more. In other embodiments, the recovery of a natriuretic peptide from a solution stored in a container is about 95% when stored at a temperature from about 4 to about 15° C. for a period of about 4 days or more. In additional embodiments, the recovery of a natriuretic peptide from a solution stored in a container is about 97% when stored at a temperature from about 4 to about 15° C. for a period of about 4 days or more. In certain embodiments, the purity of a natriuretic peptide recovered from a solution stored in a container is about 92% when stored at a temperature from about 4 to about 15° C. for a period of about 4 days or more. In certain other embodiments, the purity of a natriuretic peptide recovered from a solution stored in a container is about 95% when stored at a temperature from about 4 to about 15° C. for a period of about 4 days or more. In further embodiments, the purity of a natriuretic peptide recovered from a solution stored in a container is about 97% when stored at a temperature from about 4 to about 15° C. for a period of about 4 days or more. In certain embodiments, the container has a glass surface.

To improve stability, the headspace of any container containing or storing a composition containing a peptide of the invention can be flushed with nitrogen or another inert gas. Further, the reservoir of any provisioning apparatus can similarly be flushed with nitrogen or an inert gas.

It will be apparent to one skilled in the art that various combinations and/or modifications and variations can be made. Features illustrated or described as being part of one embodiment may be used on another embodiment to yield a still further embodiment.

### Example 1

### Preparation of Tris Buffer with 0.25% (wt.) Meta-Cresol

16.0 g glycerol, 6.05 g tris-(hydroxymethyl)-aminomethane (“Tris”), 2.50 g meta cresol were mixed in a 1.00 L volumetric flask. Approximately 900 mL nanopure water was added to the volumetric flask and the mixture was magnetically stirred to reach complete dissolution. 4 normal hydrochloric acid was used to adjust pH to 7.3 at 25° C. Then, the flask was filled to 1 L mark with nanopure water. The pH was rechecked and verified to be 7.3 at 25° C. The pH 7.3 Tris buffer was stored at 2-8° C. until use.

In any embodiment, the Tris buffer can be degassed by purging with nitrogen or other inert gas prior to use. Further, the Tris buffer can be stored in container where any headspace in the container has been purged with nitrogen or another inert gas.

Tris and glycerol were acquired from Sigma-Aldrich (St. Louis). Meta-cresol was obtained from Harrell Industries.

### Example 2

### Preparation of Phosphate Buffered Saline (PBS) with 0.25% (wt.) Meta-Cresol

7.50 g Sodium Chloride, 1.80 g sodium dihydrogenphosphate, 1.30 g sodium monohydrogenphosphate and 2.5 g meta-cresol were mixed in 1.00 L volumetric flask. After adding approximately 900 mL nanopure water, the mixture was magnetically stirred to reach complete dissolution. 1 normal aqueous sodium hydroxide solution was titrated to adjust pH to 7.4 at 25° C. Then, the flask was filled to 1 L mark with nanopure water. The pH was rechecked and verified to be 7.4 at 25° C. The PBS, PH 7.4, was store at 2-8° C. until use.

In any embodiment, the PBS can be degassed by purging with nitrogen or other inert gas prior to use. Further, the PBS can be stored in container where any headspace in the container has been purged with nitrogen or another inert gas.

Sodium chloride, sodium dihydrogenphosphate, and sodium monohydrogenphosphate were acquired from Sigma-Aldrich (St. Louis). Meta-cresol was obtained from Hurral Industry.

### Example 3

### Stability of Vessel Dilator (VD) in Tris Buffer, PBS or Saline

The stability of VD in solution at 37° C. was assessed using high performance liquid chromatography (HPLC). In all examples disclosed herein, detection of proteins or peptides using HPLC was done by UV absorbance at 214 nm. Percent recovery was calculated by comparing the main chromatographic peak area generated by VD to a control sample of the same solution maintained at 4° C. at Day 0 (day of formulation of the solutions). Purity was calculated by dividing the peak area of the main peak observed during HPLC by the total chromatographic peak area observed.

Stock solutions of VD at a concentration of 7.2, 0.72 and 0.072 mg/mL were prepared by dissolving lyophilized VD in the Tris buffer and the PBS described in Examples 1 and 2. Identical aliquots of each VD stock solution were placed in glass vials and stored at either 37° C. or at 4° C. for use as a control. An additional stock solution of VD in saline (Hospira, Lake Forrest, Ill.) was prepared. The purity of the solutions was checked shortly after formation of the solution (Day 0) and after storage at 37° C. for 14 days using the procedure described above. The recovery of protein, peptide or polypeptide from the solutions stored at 37° C. was determined after storage at 37° C. for 14 days using the procedures described above. Samples in Examples described herein were analyzed by Reverse Phase (RP)-HPLC. The RP-HPLC analysis procedure is described in Table 1 below.

To improve stability, the headspace of any container containing or storing a composition containing a natriuretic peptide can be flushed with nitrogen or another inert gas. Further, the reservoir of any provisioning apparatus can similarly be flushed with nitrogen or an inert gas.

TABLE 1
Reverse Phase (RP)-HPLC method

| Parameter | Description | | |
|---|---|---|---|
| HPLC instrument | Waters 2695 with 2998 PDA Detector | | |
| Column | Grace Vydac Peptide and Protein Column, | | |
| | C18, 5 μm, 4.6 × 250 mm or equivalent | | |
| Software | Waters Empower 2 | | |
| Mobile Phase A (MPA) | 0.1% TFA in Purified Water | | |
| Mobile Phase B (MPB) | 0.1% TFA in (60:40) Acetonitrile:Water | | |
| Flow Rate | 1.0 mL/minute | | |
| Detection Wavelength | 214 nm | | |
| Column Temperature | 60° C. | | |
| Autosampler | 5° C. | | |
| Temperature | | | |
| Run Time | 45 Minutes | | |
| Injection Volume | 10 μL | | |
| | Time | | |
| HPLC Gradient: | (minutes) | % MPA | % MPB |
| | 0 | 100 | 0 |
| | 40 | 0 | 100 |
| | 40.1 | 100 | 0 |
| | 45 | 100 | 0 |

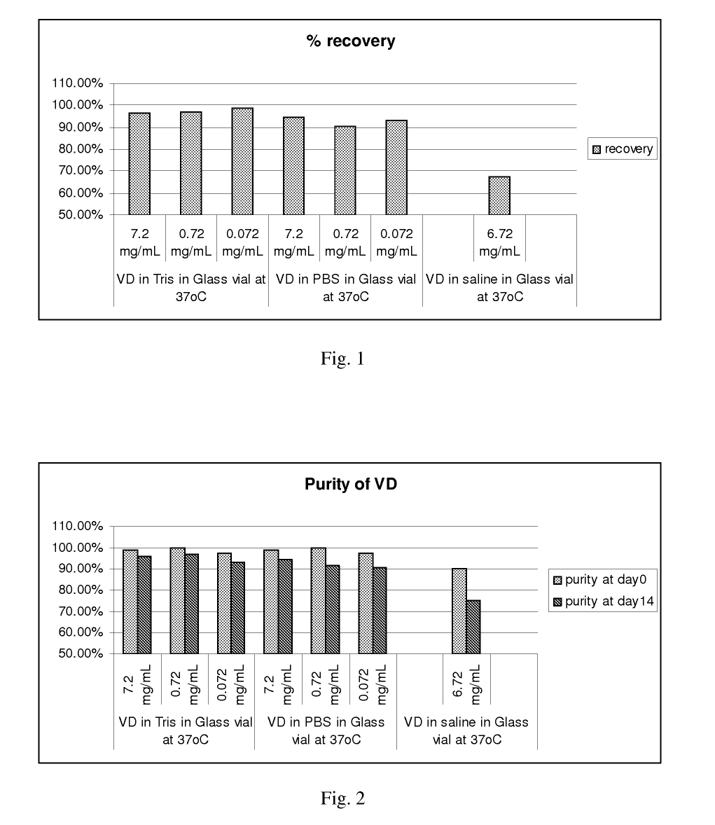

The results of the recovery and purity determinations are presented in Table 2 and provided in graphical form in FIGS. 1 and 2. As shown in Table 2, the purity of VD present in solution after 14 days of storage at 37° C. in the Tris buffer, and hence overall stability, was unexpectedly superior to storage in either the PBS or VD buffer. The purities at day 0 immediately after formation of the stock solutions in Tris buffer and PBS were indistinguishable. However, after 14 days of storage at 37° C., there were significant changes in the purity of the VD in the solutions. None of the VD in PBS solutions had a purity greater than 95% after 14 days of storage at 37° C. The VD in PBS solution at a concentration of 0.072 mg/mL had a purity less than 91%, meaning that close to 10% of the VD had degraded after 14 days.

The VD in Tris solutions showed only a slight degradation of the VD peptide after 14 days of storage at 37° C. Specifically, the VD in Tris solutions at concentrations of 7.2 and 0.72 mg/mL had purities well in excess of about 95%. Specifically, greater than about 96% and greater than about 97%, respectively. The stability, as measured by purity, for the 0.072 mg/mL was not observed to be as satisfactory.

The VD in Tris solutions also showed superior recovery compared to VD in PBS solutions. All the VD in Tris solutions exhibit a recovery greater than about 95% after 14 days of storage at 37° C., including greater than about 96%, greater than about 97% and greater than about 98%. All of the VD in PBS solutions exhibited a recovery less than about 95% after 14 days of storage at 37° C. The relatively concentrated VD in Tris solution at 0.72 mg/mL had a recovery of about 90% of the original mass of the peptide after 14 days of storage at 37° C. The VD in saline solution exhibits particularly poor stability characteristics. After 14 days of storage at 37° C., a recovery of 67.5% indicated that close to a third of the mass of VD was lost from solution. Further, the purity of 75.2% indicated that close to a quarter of the remaining mass from the VD peptide is degraded into a chemical identity other than full, unmodified VD peptide.

In certain embodiments, the recovery of a natriuretic peptide from a solution is about 95% or more when stored at a temperature from about 25 to about 45° C. for a period of about 14 days or more. In other embodiments, the recovery of a natriuretic peptide from a solution is about 97% or more when stored at a temperature from about 25 to about 45° C. for a period of about 14 days or more. In certain embodiments, the purity of a natriuretic peptide present in a solution is about 92% or more when stored at a temperature from about 25 to about 45° C. for a period of about 14 days or more. In certain other embodiments, the purity of a natriuretic peptide present in a solution is about 96% or more when stored at a temperature from about 25 to about 45° C. for a period of about 14 days or more. In other embodiments, the purity of a natriuretic peptide present in a solution is about 97% or more when stored at a temperature from about 25 to about 45° C. for a period of about 14 days or more.

In certain embodiments, the change in relative purity of a natriuretic peptide present a solution is about 10% or less when stored at a temperature from about 25 to about 45° C. for a period of about 14 days or more. In certain other embodiments, the purity of a natriuretic peptide present in a solution is about 8% or less when stored at a temperature from about 25 to about 45° C. for a period of about 14 days or more. In other embodiments, the purity of a natriuretic peptide present in a solution is about 5% or more when stored at a temperature from about 25 to about 45° C. for a period of about 14 days or more. In still other embodiments, the purity of a natriuretic peptide present in a solution is about 3% or more when stored at a temperature from about 25 to about 45° C. for a period of about 14 days or more.

TABLE 2
Recovery and purity of VD in Tris buffer, PBS or saline

| | | Recovery | purity at | purity at |
|---|---|---|---|---|
| | concentration | at day 14 | day 0 | day 14 |
| VD in Tris in Glass | 7.2 mg/mL | 96.47% | 99.02% | 96.12% |
| vial at 37° C. | 0.72 mg/mL | 96.66% | 99.74% | 97.04% |
| | 0.072 mg/mL | 98.96% | 97.42% | 93.10% |
| VD in PBS in Glass | 7.2 mg/mL | 94.56% | 99.02% | 94.65% |
| vial at 37° C. | 0.72 mg/mL | 90.49% | 99.74% | 91.66% |
| | 0.072 mg/mL | 93.21% | 97.42% | 90.73% |
| VD in saline in Glass | 6.72 mg/mL | 67.5% | 90.1% | 75.2% |
| vial at 37° C. | | | | |

### Example 4

### Stability of 1.25 mg/mL VD in Tris Buffer Delivered from a Provisioning Apparatus

The suitability for delivery of a 1.25 mg/mL solution of VD prepared in the Tris buffer of Example 1 was evaluated by delivery from Medtronic MiniMed® Paradigm® pumps using a MiniMed 3.0 mL reservoir (MMT-332A). The pump reservoirs were filled by connection to MMT-296 Quick-Set™ infusion sets and primed with the 1.25 mg/mL VD in Tris buffer formulation. Upon filling the pumps with the formulation of VD, all air bubbles were removed from the reservoirs. The solution pumped by the pumps was collected in non-vented 4 mL glass vials that were seated with Teflon™ lined septa that were pierced with Quick-set™ infusion sets. The volume of the vials was at least 10 times the expected pumping volume so that the pressure changes in the vials were minimal and venting was unnecessary. The Paradigm® pumps containing the 1.25 mg/mL solution of VD in the Tris buffer were equilibrated to a temperature of 37° C. to simulate conditions present due to body heat emanating from a subject and subjected to continuous agitation at 100±10 strokes/minute with a one inch shaking distance on an orbital shaker. Pumps were operated at a rate 0f 0.016 mg/hr.

Two controls were also performed. In the first control, the 1.25 mg/mL solution of VD in Tris buffer was placed in MiniMed 3.0 mL reservoirs but not actively pumped to serve as reservoir controls. All air bubbles were removed from the control reservoirs. The control reservoirs contained 1 mL of the VD in Tris buffer formulation and were incubated at 37° C. without agitation. In the second control, the 1.25 mg/mL solution of VD was placed in glass vials and maintained at either 4 or 37° C. to serve as glass controls. The glass controls were prepared by adding 200 μL of the 1.25 mg/mL solution of VD to 1.6 mL glass HPLC vials without agitation. Control as well as experimental trials using the Medtronic MiniMed® Paradigm® pump or unpumped reservoirs were performed using 5 separate trials to calculate a standard deviation (SD) where indicated.

Purity was calculated by analysis using RP-HPLC, as described above in Example 3. Briefly, recovery was calculated by dividing the peak area observed for the main peak associated with VD by the peak are obtained for the glass control at 4° C. on Day 0. Purity was calculated by dividing the main peak area for VD by the total observed chromatographic peak area. For the pump samples, the solution pumped through the catheter of the pump into the collection vials was measured on a daily basis for 6 days; the remaining solutions (residual) in the 3 mL reservoir of the pumps after the 6-day period were also analyzed.

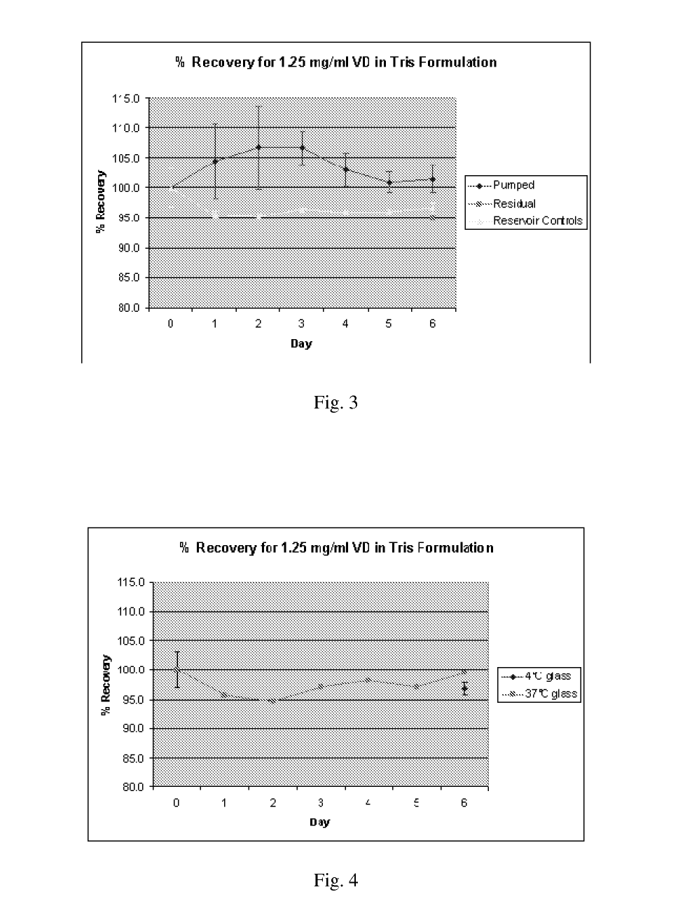

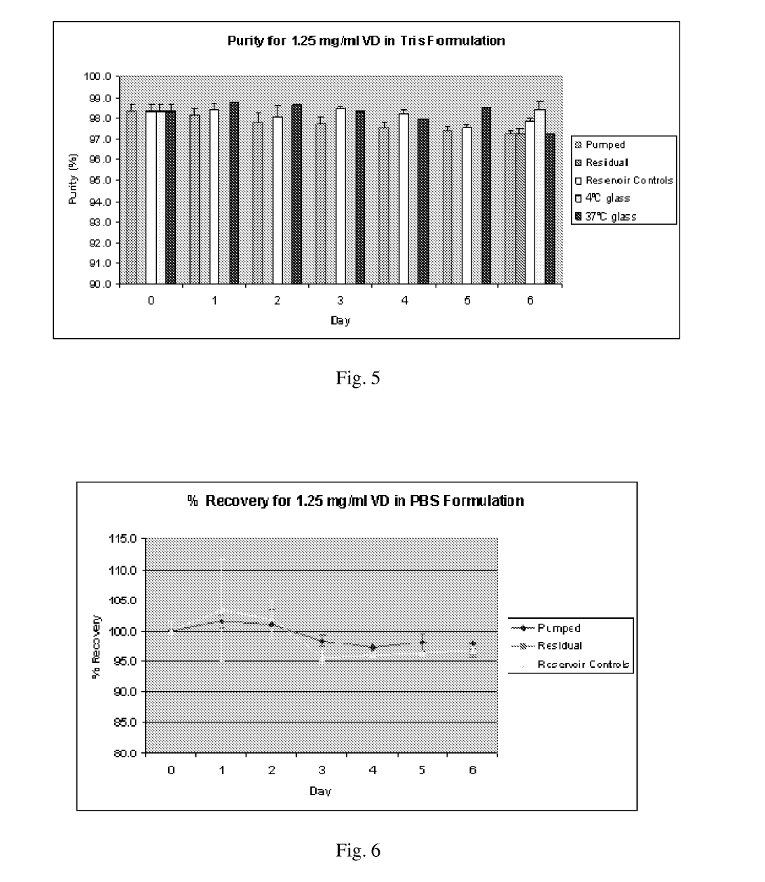

The results for the recovery of VD from the Paradigm® pumps are presented in Table 3 and FIGS. 3 and 4 along with results from the controls employing the unpumped reservoirs and glass vials. The results for purity for the same samples are reported in Table 4 and FIG. 5.

The 1.25 mg/mL VD in Tris buffer composition exhibited good stability characteristics. The recovery for the 1.25 mg/mL VD in Tris buffer was stable over the 6 day period measured for the experimental pumped samples and for the control samples. Interestingly, the recovery from the experimental pumped samples was consistently higher than for the unpumped controls and the glass vials controls. The purity of the 1.25 mg/mL VD in Tris buffer was also stable over the 6 day period. In all experimental and control samples measured, purity did not decrease by more than about 1%. Recovery is reported as a percentage of the peptide concentration measured at day 0; the residual data point is for the 1.25 mg/mL VD in Tris buffer remaining in the pump reservoir after the 6-day experiment.

The small change in purity of the pumped samples and the reservoir controls (unpumped) over the 6 days was similar to that for the glass controls at the same temperature. The purity of the residual sample after remaining in the pumped reservoirs after 6 days remained above about 97%. The higher amount of degradation (lower purity) at 37° C. in glass vials compared to 4° C. follows the expected temperature dependence.

TABLE 3
Recovery of 1.25 mg/mL VD in Tris buffer solution
from Paradigm ® pumps and controls

| | | Reservoir | | |
|---|---|---|---|---|
| | Pumped Samples | Controls | 4° C. glass | 37° C. glass |
| Day | % Recovery | % Recovery | % Recovery | % Recovery |
| 0 | 100 ± 3.1 | 100 ± 3.1 | 100 ± 3.1 | 100 ± 3.1 |
| 1 | 104.4 ± 6.2 | 95.5 ± 0.7 | | 95.7 |
| 2 | 106.7 ± 7.0 | 95.2 ± 0.4 | | 94.7 |
| 3 | 106.7 ± 2.8 | 96.4 ± 0.3 | | 97.0 |
| 4 | 103.0 ± 2.7 | 95.9 ± 0.3 | | 98.3 |
| 5 | 100.9 ± 1.8 | 95.9 ± 0.5 | | 97.0 |
| 6 | 101.5 ± 2.3 | 96.7 ± 0.9 | 96.6 ± 1.1 | 99.5 |
| residual | 94.9 ± 0.4 | | | |

TABLE 4
Purity of 1.25 mg/mL VD in Tris buffer solution
from Paradigm ® pumps and controls

| | | Reservoir | | |
|---|---|---|---|---|
| | Pump Samples | Controls | 4° C. glass | 37° C. glass |
| Day | Purity (%) | Purity (%) | Purity (%) | Purity (%) |
| 0 | 98.3 ± 0.3 | 98.3 ± 0.3 | 98.3 ± 0.3 | 98.3 ± 0.3 |
| 1 | 98.2 ± 0.3 | 98.38 ± 0.3 | | 98.7 |
| 2 | 97.8 ± 0.5 | 98.03 ± 0.5 | | 98.6 |
| 3 | 97.7 ± 0.3 | 98.44 ± 0.1 | | 98.4 |
| 4 | 97.5 ± 0.3 | 98.21 ± 0.2 | | 98.0 |
| 5 | 97.4 ± 0.2 | 97.5 ± 0.2 | | 98.5 |
| 6 | 97.3 ± 0.1 | 97.85 ± 0.2 | 98.4 ± 0.4 | 97.3 |
| residual | 97.2 ± 0.2 | | | |

### Example 5

### Stability of 1.25 mg/mL VD in PBS Delivered from a Provisioning Apparatus

The suitability for delivery of a 1.25 mg/mL solution of VD prepared in the PBS of Example 2 was evaluated by delivery from Medtronic MiniMed® Paradigm® pumps using a MiniMed 3.0 mL reservoir (MMT-332A). The procedure to Evaluate stability in samples pumped from the Paradigm® pumps and reservoir and glass controls were the same as for Example 4. Briefly, the Paradigm® pumps containing the 1.25 mg/mL solution of VD in the PBS were equilibrated to a temperature of 37° C. to simulate conditions present due to body heat emanating from a subject and agitated as described. The pump or provisioning apparatus was operated at a rate of 0.016 mL/hr. The content of the solution passing through the catheter was measured by RP-HPLC from 5 separate pumps to calculate an average value with a standard deviation (SD) where indicated. The content of the solution remaining in the reservoir after 6 days was also measured. Controls in unpumped reservoirs and glass were also performed as described in Example 4.

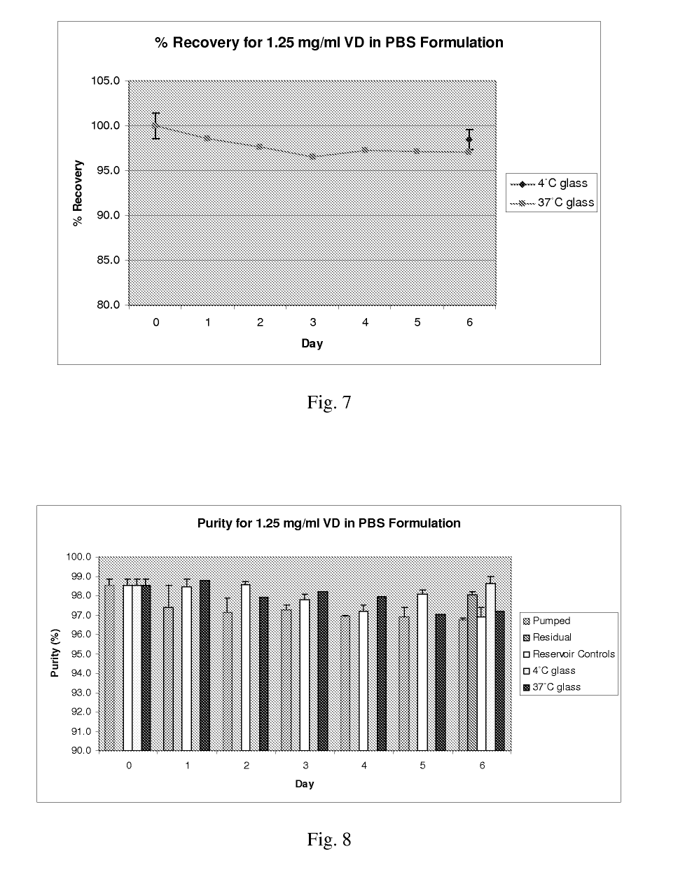

The results for the recovery of VD from the Paradigm® pumps are presented in Table 5 and FIGS. 6 and 7 along with results from the controls employing the unpumped reservoirs and glass vials. The results for purity for the same samples are reported in Table 6 and FIG. 8. The recovery of VD peptide remained about 95% at 37° C. over 6 days for the experimental pump samples, the reservoir controls and the glass vial controls. For the pumped samples, recovery appeared to be stable for the experimental pumped samples remained near 100% for the first two days, where a drop-off in recovery occurred after about 3 to 4 days. A similar pattern was observed for the reservoir controls (unpumped) as well.

The small change in purity of the pumped samples (Table 6 and FIG. 8) over 6 days was similar to that for the glass controls at the same temperature. The purity of the residual samples in the pump reservoirs remained above about 98% after six days. The higher amount of degradation (lower purity) at 37° C. in glass vials compared to 4° C. follows the expected temperature dependence.

TABLE 5
Recovery of 1.25 mg/mL VD in PBS solution from
Paradigm ® pumps and controls

| | | Reservoir | | |
|---|---|---|---|---|
| | Pumped Samples | Controls | 4° C. glass | 37° C. glass |
| Day | % Recovery | % Recovery | % Recovery | % Recovery |
| 0 | 100 ± 1.4 | 100 ± 1.4 | 100 ± 1.4 | 100 ± 1.4 |
| 1 | 101.5 ± 1.0 | 103.4 ± 8.2 | | 98.6 |
| 2 | 101.0 ± 2.4 | 101.9 ± 3.2 | | 97.6 |
| 3 | 98.3 ± 0.9 | 95.7 ± 1.0 | | 96.5 |
| 4 | 97.2 ± 0.7 | 96.1 ± 0.4 | | 97.3 |
| 5 | 98.1 ± 1.4 | 96.3 ± 0.2 | | 97.2 |
| 6 | 98.0 ± 0.2 | 96.9 ± 0.3 | 98.5 ± 1.1 | 97.0 |
| residual | 96.4 ± 0.7 | | | |

TABLE 6
Purity of 1.25 mg/mL VD in PBS solution from
Paradigm ® pumps and controls

| | | Reservoir | | |
|---|---|---|---|---|
| | Pump Samples | Controls | 4° C. glass | 37° C. glass |
| Day | Purity (%) | Purity (%) | Purity (%) | Purity (%) |
| 0 | 98.5 ± 0.4 | 98.5 ± 0.4 | 98.5 ± 0.4 | 98.5 ± 0.4 |
| 1 | 97.4 ± 1.1 | 98.5 ± 0.4 | | 98.8 |
| 2 | 97.1 ± 0.8 | 98.6 ± 0.2 | | 97.9 |
| 3 | 97.2 ± 0.3 | 97.8 ± 0.3 | | 98.2 |
| 4 | 96.9 ± 0.0 | 97.2 ± 0.3 | | 98.0 |
| 5 | 96.9 ± 0.5 | 98.1 ± 0.2 | | 97.0 |
| 6 | 96.7 ± 0.1 | 96.9 ± 0.5 | 98.6 ± 0.4 | 97.2 |
| residual | 98.0 ± 0.2 | | | |

### Example 6

### Stability of 15 mg/mL VD in Tris Buffer Delivered from a Provisioning Apparatus

The suitability for delivery of a 15 mg/mL solution of VD prepared in the Tris buffer of Example 1 was evaluated by delivery from Medtronic MiniMed® Paradigm® pumps using a MiniMed 3.0 mL reservoir (MMT-332A). Stability was evaluated in the same fashion as Example 4, except a 15 mg/mL formulation of VD in Tris buffer was used instead of a 1.25 mg/mL formulation in Tris buffer. The procedure to evaluate stability in samples pumped from the Paradigm® pumps and reservoir and glass controls were the same as for Example 4. Briefly, the Paradigm® pumps containing the 15 mg/mL solution of VD in the Tris buffer were equilibrated to a temperature of 37° C. to simulate conditions present due to body heat emanating from a subject and agitated as described. The pump or provisioning apparatus was operated at a rate of 0.016 mL/hr. The content of the solution passing through the catheter was measured by RP-HPLC from 5 separate pumps to calculate an average value with a standard deviation (SD) when indicated. The content of the solution remaining in the reservoir after 6 days was also measured. Controls in unpumped reservoirs and glass were also performed as described in Example 4.

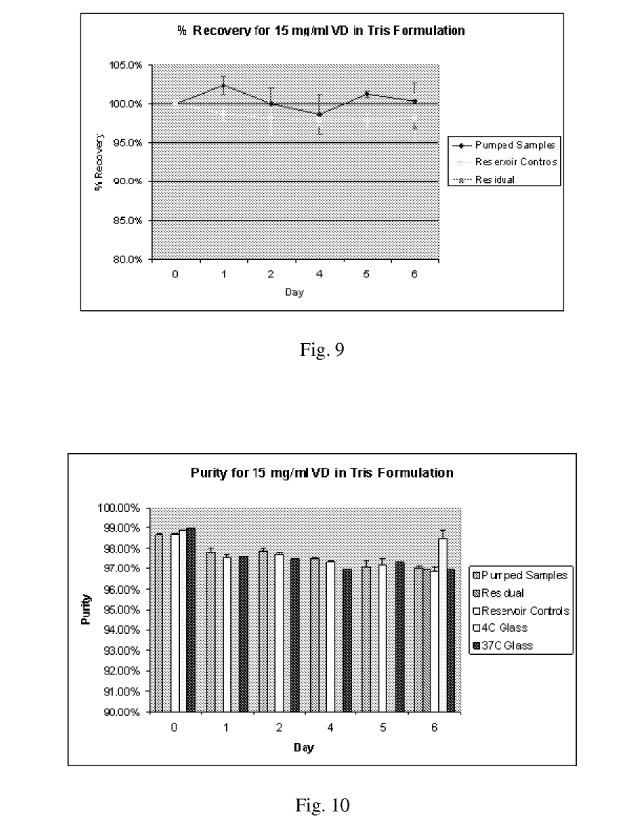

The results for the recovery of VD from the Paradigm® pumps are presented in Table 7 and FIG. 9 along with results from the controls employing the unpumped reservoirs. The results for purity for the same samples and the glass vial controls are reported in Table 8 and FIG. 10. The recovery of the pumped VD peptide remained near 100% at 37° C. over 6 days for the experimental pump samples. Recovery from the control unpumped reservoir samples appeared to be consistently lower than the experimental pumped samples. The recovery of the VD peptide remained above about 97% over the 6 days.

The small change in purity of the pumped samples (Table 8 and FIG. 10) over 6 days was similar to that for the glass controls at the same temperature. The purity of the residual samples in the pump reservoirs remained above about 97% after six days. The higher amount of degradation (lower purity) at 37° C. in glass vials compared to 4° C. follows the expected temperature dependence.

TABLE 7
Recovery of 15 mg/mL VD in Tris buffer from
Paradigm ® pumps and controls

| | Pumped Samples | Reservoir Controls |
|---|---|---|
| Day | % Recovery | % Recovery |
| 0 | 100.0 ± 0.5 | 100 ± 0.5 |
| 1 | 102.3 ± 1.3 | 98.8 ± 0.9 |
| 2 | 99.9 ± 2.0 | 98.2 ± 1.9 |
| 4 | 98.6 ± 2.5 | 98.0 ± 0.8 |
| 5 | 101.2 ± 0.4 | 97.9 ± 0.9 |
| 6 | 100.3 ± 2.4 | 98.1 ± 3.1 |
| residual | 97.2 ± 0.6 | |

TABLE 8
Purity of 15 mg/mL VD in Tris buffer from
Paradigm ® pumps and controls

| | | Reservoir | | |
|---|---|---|---|---|
| | Pump Samples | Controls | 4° C. glass | 37° C. glass |
| Day | Purity (%) | Purity (%) | Purity (%) | Purity (%) |
| 0 | 98.7 ± 0.1 | 98.7 ± 0.1 | 98.9 | 99.0 |
| 1 | 97.9 ± 0.2 | 97.6 ± 0.1 | | 97.6 |
| 2 | 97.9 ± 0.1 | 97.7 ± 0.1 | | 97.5 |
| 4 | 97.5 ± 0.1 | 97.4 ± 0.1 | | 97.0 |
| 5 | 97.1 ± 0.4 | 97.2 ± 0.3 | | 97.3 |
| 6 | 97.0 ± 0.1 | 96.9 ± 0.2 | 98.5 ± 0.4 | 97.0 |
| residual | 97.0 ± 0.1 | | | |

### Example 7

### Stability of 15 mg/mL VD in PBS Delivered from a Provisioning Apparatus

The suitability for delivery of a 15 mg/mL solution of VD prepared in the PBS of Example 2 was evaluated by delivery from Medtronic MiniMed® Paradigm® pumps using a MiniMed 3.0 mL reservoir (MMT-332A). Stability was evaluated in the same fashion as in Examples 4 and 5, except a 15 mg/mL formulation of VD in PBS was used in the Paradigm® pumps and controls. The procedure to evaluate stability in samples pumped from the Paradigm® pumps and reservoir and glass controls were the same as for Example 4. Briefly, the Paradigm® pumps containing the 15 mg/mL solution of VD in the PBS were equilibrated to a temperature of 37° C. to simulate conditions present due to body heat emanating from a subject and agitated as described. The pump or provisioning apparatus was operated at a rate of 0.016 mL/hr. The content of the solution passing through the catheter was measured by RP-HPLC from 5 separate pumps to calculate an average value with a standard deviation (SD) when indicated. The content of the solution remaining in the reservoir after 6 days was also measured. Controls in unpumped reservoirs and glass were also performed as described in Example 4.

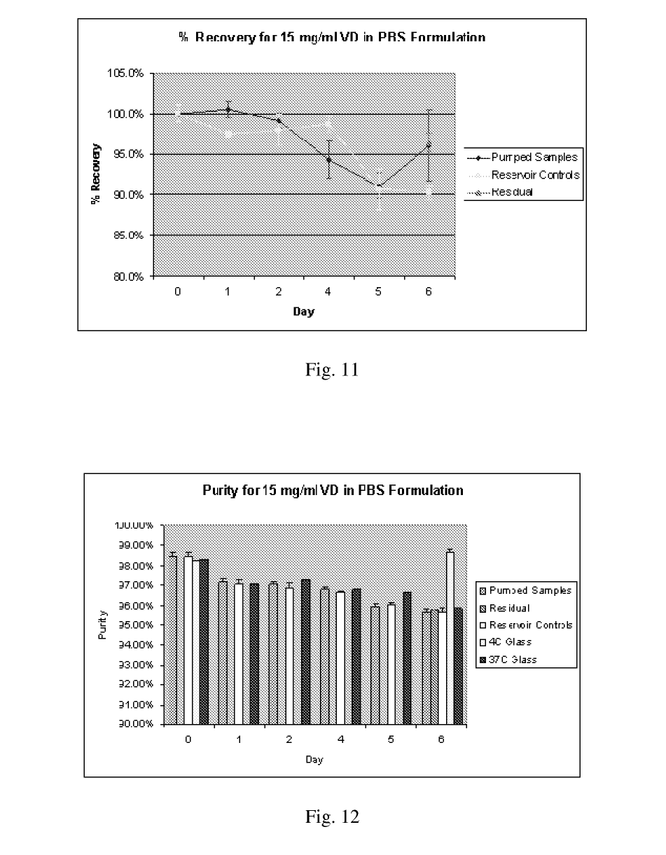

The results for the recovery of VD from the Paradigm® pumps are presented in Table 9 and FIG. 11 along with results from the controls employing the unpumped reservoirs. The results for purity for the same samples and the glass vial controls are reported in Table 10 and FIG. 12. The recovery of the pumped VD peptide remained above 95% at 37° C. over 6 days for the experimental pump samples. Recovery from the control unpumped reservoir samples appeared to be significantly lower than the experimental pumped samples in Table 9. After 6 days, the recovery from the unpumped reservoir control decreased to about 90%. In the unpumped reservoir control, the decrease in recovery appeared to accelerate after about 4 days.

The purity of the pumped samples (Table 10 and FIG. 12) over 6 days was similar to that for the glass controls at the same temperature. A drop in purity occurred over the 6-day period; however, the purity of the residual samples in the pump reservoirs remained above about 95% after 6 days. The difference in the purity of the pumped samples and the unpumped reservoir samples was small over the 6-day period. The higher amount of degradation (lower purity) at 37° C. in glass vials compared to 4° C. follows the expected temperature dependence.

TABLE 9
Recovery of 15 mg/mL VD in PBS solution from
Paradigm ® pumps and controls

| | Pumped Samples | Reservoir Controls |
|---|---|---|
| Day | % Recovery | % Recovery |
| 0 | 100.0 ± 0.9 | 100 ± 0.9 |
| 1 | 100.5 ± 0.9 | 97.5 ± 0.4 |
| 2 | 99.1 ± 0.9 | 98.0 ± 1.9 |
| 4 | 94.3 ± 2.2 | 98.7 ± 0.9 |
| 5 | 91.0 ± 1.6 | 90.7 ± 2.5 |
| 6 | 96.1 ± 4.3 | 90.3 ± 0.9 |
| residual | 96.5 ± 1.1 | |

TABLE 10
Purity of 15 mg/mL VD in PBS solution from
Paradigm ® pumps and controls

| | | Reservoir | | |
|---|---|---|---|---|
| | Pump Samples | Controls | 4° C. glass | 37° C. glass |
| Day | Purity (%) | Purity (%) | Purity (%) | Purity (%) |
| 0 | 98.4 ± 0.2 | 98.4 ± 0.2 | 98.3 | 98.3 |
| 1 | 97.2 ± 0.2 | 97.1 ± 0.3 | | 97.1 |
| 2 | 97.1 ± 0.1 | 96.9 ± 0.3 | | 97.3 |
| 4 | 96.8 ± 0.1 | 96.7 ± 0.1 | | 96.8 |
| 5 | 95.9 ± 0.2 | 96.0 ± 0.1 | | 96.7 |
| 6 | 95.7 ± 0.2 | 95.7 ± 0.1 | 98.7 ± 0.1 | 95.9 |
| residual | 95.8 ± 0.0 | | | |

In certain embodiments, the change in relative purity of a natriuretic peptide in a composition administered by a provisioning apparatus over a course of at least 6 days is about 5% or less when at a temperature from about 25 to about 45° C. In certain other embodiments, the change in relative purity of a natriuretic peptide in a composition administered by a provisioning apparatus over a course of at least 6 days is about 3% or less when at a temperature from about 25 to about 45° C. In other embodiments, the change in relative purity of a natriuretic peptide in a composition administered by a provisioning apparatus over a course of at least 6 days is about 2% or less when at a temperature from about 25 to about 45° C. In certain embodiments, the change in relative purity of a natriuretic peptide in a composition stored in a container or in a provisioning apparatus over a course of at least 6 days is about 15% or less when at a temperature from about 25 to about 45° C. In certain other embodiments, the change in relative purity of a natriuretic peptide in a composition stored in a container or in a provisioning apparatus over a course of at least 6 days is about 10% or less when at a temperature from about 25 to about 45° C. In other embodiments, the change in relative purity of a natriuretic peptide in a composition stored in a container or in a provisioning apparatus over a course of at least 6 days is about 5% or less when at a temperature from about 25 to about 45° C.

In certain embodiments, the pump or provisioning apparatus delivers a composition at a rate from about 0.005 to about 0.04 mL/hr. In certain other embodiments, the pump or provisioning apparatus delivers a composition at a rate from about 0.01 to about 0.025 mL/hr. In other embodiments, the pump or provisioning apparatus delivers a composition at a rate from about 0.012 to about 0.02 mL/hr.

### Example 8

### Physiological Response to Administration of VD by Subcutaneous Infusion in a Rat Model

The pharmacodynamic effects of VD were investigated in a rat model. Forty male Dahl/SS rats were shipped to the animal facilities at PhysioGenix, Inc. (Milwaukee, Wis.). The rats were maintained on a low-salt diet and allowed to acclimate. After acclimation, animals had baseline parameters collected while on the low-salt diet. Animals were then randomly assigned to one of 4 groups (10 animals per group):

1. Vehicle Control; low-salt diet, n=10
2. Vehicle Control; 4% salt diet, n=10
3. Vessel dilator, 100 ng/kg/min infusion of VD, 4% salt diet, n=10
4. Vessel dilator, 300 ng/kg/min infusion of VD, 4% salt diet, n=10

Lyophilized VD peptide (Bachem) was reconstituted in a Tris buffer having the same composition as the Tris buffer of Example 1. The vehicle control groups were infused with a citrate-mannitol-saline buffer (0.66 mg/mL citric acid, 6.43 mg/mL sodium citrate, 40 mg/mL mannitol, 9 mg/mL NaCl). The animals were on a Teklad 7034 (low-salt) diet or Dyets AIN-76A 4% salt diet, as indicated, throughout a 6 week course of the study and had free access to water. All animals receiving subcutaneous (SQ) infusion of VD were on the high-salt diet.

Alzet® minipumps (Durect, Corp.) were surgically implanted on Days 1, 15, and 29 of the study to maintain continuous vehicle or drug dispensing at the desired dose for a total period of 6 weeks. At the end of 6 weeks, the serum content of Prostaglandin E₂ (PGE2) was measured.

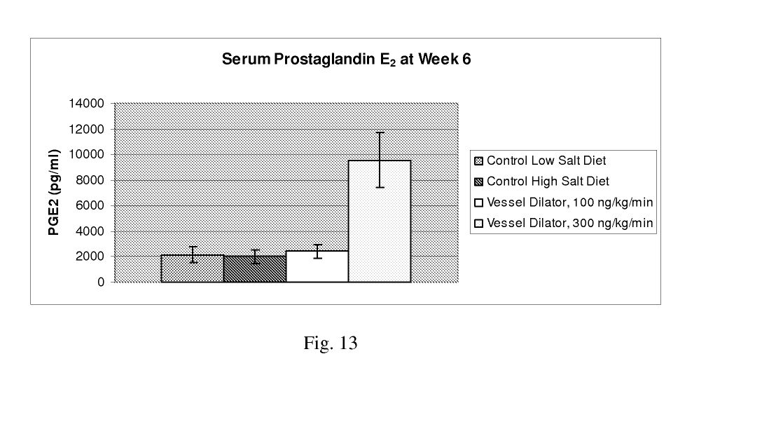

FIG. 13 shows the increase in PGE2 concentration (pg/mL) in the serum for the rat model after 6 weeks. Standard error is shown by the error bars on FIG. 13. As indicated on FIG. 13, the PGE2 concentration for the control groups on both the low- and high-salt diets are similar the experimental group receiving SQ infusion of VD at a rate of 100 ng/kg·min. However, the experimental group receiving SQ infusion of VD at a rate of 300 ng/kg·min showed a concentration of PGE2 significantly higher than for any of the other 3 groups. The difference in average PGE2 concentration between the experimental group infused with VD at a rate of 300 ng/kg·min and both control groups was statistically significant (p<0.05).

An increase in PGE2 is suggestive of the mechanism of action of Vessel Dilator. Without being limited to any one theory, PGE2 could modulate sodium transport in vivo and may contribute to the final regulation of sodium excretion by reducing tubular sodium transport at the level of the kidney, thereby creating a natriuretic effect. In administration by SQ infusion, the therapeutic agent (e.g. VD) is administered beneath the skin and into subcutaneous tissue. In general, the absorption rate from SQ delivery is slower than from the intramuscular site. Hence, SQ administration may be better suited for long-term therapy. Without being limited to any one theory, the major barrier to absorption from the intramuscular or subcutaneous sites is believed to be the capillary endothelial membrane or cell wall. Nonetheless, SQ delivery can be an advantageous route of administration for achieving prolonged therapeutic effect. The effect of SQ delivery of VD on PGE2 plasma concentration in the rat model, as described in FIG. 13, indicates that VD in the formulations of the present invention can be successfully delivered by SQ infusion to have a physiological effect.

What is claimed is:

1. A protein composition, comprising: a peptide selected from the group consisting of atrial natriuretic peptide (ANP), vessel dilator (VD), kaliuretic peptide (KP), atrial long-acting natriuretic peptide (LANP), urodilatin (URO), and variants thereof; one or more buffers selected from the group consisting of tris(hydroxymethyl)aminomethane and a phosphate buffer; meta-cresol; and water.

2. The protein composition of claim 1, wherein the peptide is selected from the group consisting of SEQ ID No.'s 3-7.

3. The protein composition of claim 1, wherein meta-cresol is present at a concentration from about 0.15 to about 0.315% by weight.

4. The protein composition of claim 1, wherein the concentration of tris(hydroxymethyl)aminomethane is from about 5 to about 100 mM.

5. The protein composition of claim 1, wherein the concentration of tris(hydroxymethyl)aminomethane is from about 10 to about 75 mM.

6. The protein composition of claim 1, wherein the protein composition has a pH from about 6.5 to about 7.6.

7. The protein composition of claim 1, wherein the protein composition has a composition such that the pH is from about 6.5 to about 7.6 when adjusted to a temperature of 25° C.

8. The protein composition of claim 1, wherein a concentration of the peptide is from about 0.05 to about 20 mg/mL.

9. The protein composition of claim 1, wherein the protein composition further comprises glycerol.

10. The protein composition of claim 9, wherein the protein composition further comprises from about 0.1 to about 5% glycerol by weight.

11. The protein composition of claim 9, wherein the protein composition further comprises from about 0.5 to about 2% glycerol by weight.

12. The protein composition of claim 1, wherein the protein composition comprises tris(hydroxymethyl)aminomethane at a concentration of about 50 mM, meta-cresol at a concentration of about 0.25% by weight, and further comprises about 1.6% glycerol and a pH of about 7.3.

13. The protein composition of claim 1, wherein the phosphate buffer is present at a concentration from about 0.2 to about 10 grams per liter.

14. The protein composition of claim 1, wherein the composition further comprises sodium chloride at a composition from about 2 to about 15 grams per liter.

15. The protein composition of claim 1, wherein the peptide is derived from ANP prohormone.

16. The protein composition of claim 15, wherein the peptide comprises at least about 20 amino acid residues derived from ANP prohormone.

17. The protein composition of claim 1, wherein the peptide contains an intramolecular disulfide bond between two cysteine amino acid residues comprising the same peptide.

18. The protein composition of claim 17, wherein the peptide has 15 amino acid residues that are located on the same peptide between the two cysteine amino acid residues forming the disulfide bond.

19-22. (canceled)

23. A method for stabilizing a peptide selected from the group consisting of atrial natriuretic peptide (ANP), vessel dilator (VD), kaliuretic peptide (KP), atrial long-acting natriuretic peptide (LANP), urodilatin (URO), and variants thereof in solution, comprising: dissolving the peptide in a buffer composition comprising water and one or more buffers selected from the group consisting of tris(hydroxymethyl)aminomethane and a phosphate buffer to form a protein composition wherein the buffer composition further comprises meta-cresol.

24-65. (canceled)

66. A method for administering a protein composition to a patient, comprising:
storing a protein composition in a container, the protein composition comprising a peptide selected from the group consisting of atrial natriuretic peptide (ANP), vessel dilator (VD), kaliuretic peptide (KP), atrial long-acting natriuretic peptide (LANP), urodilatin (URO), and variants thereof, one or more buffers selected from the group consisting of (hydroxymethyl)aminomethane and a phosphate buffer, and meta-cresol; and
administering the protein composition to the patient wherein the protein composition is delivered and metered to the patient using a pump or provisioning apparatus, said protein composition being stored at a temperature from about 25 to about 45° C.

67-110. (canceled)
